SEQUENCE LISTING (2 sequences)

SEQ ID NO: 1 - protein, 834 aa, Homo sapiens
MWGLGARGPDRGLLLALALGGLARAGGVEVEPGGAHGESGGFQVVTFEWAHVQDPYVIALWILVASLAKIGFHLSHKVTSVVPESALLIVLGLVLGGIVWAADHIASFTLTPTVFFFYLLPPIVLDAGYFMPNRLFFGNLGTILLYAVVGTVWNAATTGLSLYGVFLSGLMGDLQIGLLDFLLFGSLMAAVDPVAVLAVFEEVHVNEVLFIIVFGESLLNDAVTVVLYNVFESFVALGGDNVTGVDCVKGIVSFFVVSLGGTLVGVVFAFLLSLVTRFTKHVRIIEPGFVFIISYLSYLTSEMLSLSAILAITFCGICCQKYVKANISEQSATTVRYTMKMLASSAETIIFMFLGISAVNPFIWTWNTAFVLLTLVFISVYRAIGVVLQTWLLNRYRMVQLEPIDQVVLSYGGLRGAVAFALVVLLDGDKVKEKNLFVSTTIIVVFFTVIFQGLTIKPLVQWLKVKRSEHREPRLNEKLHGRAFDHILSAIEDISGQIGHNYLRDKWSHFDRKFLSRVLMRRSAQKSRDRILNVFHELNLKDAISYVAEGERRGSLAFIRSPSTDNVVNVDFTPRSSTVEASVSYLLRENVSAVCLDMQSLEQRRRSIRDAEDMVTHHTLQQYLYKPRQEYKHLYSRHELTPTEDEKQDREIFHRTMRKRLESFKSTKLGLNQNKKAAKLYKRERAQKRRNSSIPNGKLPMESPAQNFTIKEKDLELSDTEEPPNYDEEMSGGIEFLASVTKDTASDSPAGIDNPVFSPDEALDRSLLARLPPWLSPGETVVPSQRARTQIPYSPGTFCRLMPFRLSSKSVDSFLQADGPEERPPAALPESTHM

SEQ ID NO: 2 - DNA, 2777 bp, Homo sapiens
ccggcaccggagcgcgccgggtcctcgccgggtcccagccccgcgcgtcggcccagcccagccgggaccccccgcgacatgcgcgtcccagccctgcgctgagcaggcggcaatgtggggactcggggcccggggccccgaccgggggctgctgctggcgctggcgctgggcgggctggcgcgggccgggggcgtcgaggtggagcccggcggcgcgcacggcgagagcgggggcttccaggtggtcaccttcgagtgggcccacgtgcaggatccctacgtcatcgcgctctggatcctcgtggccagcttggccaagatcgggttccacctgtcccacaaggtcaccagcgtggttcccgagagcgccctgctcatcgtgctgggcctggtgctgggcggcatcgtctgggcggccgaccacatcgcgtccttcacactgacgcccaccgtcttcttcttctacctgctgccccccatcgtgctggacgccggctacttcatgcccaaccgcctcttcttcggcaacctggggaccatcctgttgtacgccgtcgtgggtaccgtgtggaacgcggccaccaccgggctgtccctctacggcgtcttcctcagtgggctcatgggcgacctgcagattgggctgctggacttcctcctgtttggcagcctcatggcggctgtggacccggtggccgtcctggccgtgtttgaggaggtccatgtcaacgaggtcctgttcatcatcgtcttcggggagtcgctgctgaacgacgcagtcaccgtggttctgtacaatgtgtttgaatctttcgtggcgctgggaggtgacaacgtgactggcgtggactgcgtgaagggcatagtgtccttcttcgtggtgagcctggggggcacgctggtgggggtggtcttcgccttcctgctgtcgctggtgacgcgcttcaccaagcatgtgcgtatcatcgagcccggcttcgtgttcatcatctcctacctgtcctacctgacgtccgagatgctgtcgctgtcggccatcctcgccatcaccttctgtggcatctgctgtcagaagtatgtgaaggccaacatctcggagcagtcggccaccaccgtgcgctacaccatgaagatgctggccagcagcgccgagaccatcatcttcatgttcctgggtatctcggccgtgaacccgttcatctggacctggaacacggccttcgtgctcctgacgctggtcttcatctccgtgtaccgggccatcggtgtggtcctgcagacctggcttctgaaccgctaccgcatggtgcagctggagcccattgaccaggtggtcctgtcctacgggggcctgcgcggggccgtggcctttgccctggtggtgcttctggatggagacaaggtcaaggagaagaacctgttcgtcagcaccaccatcatcgtagtgttcttcaccgtcatcttccagggcctgaccatcaagcctctggtgcagtggctgaaggtgaagaggagcgagcaccgggaacctcggctcaacgagaagctgcacgggcgcgctttcgaccacatcctctcggccatcgaggacatatccggacagatcgggcacaattatctcagagacaagtggtcccacttcgacaggaagttcctcagcagggtcctcatgagacggtcggcccagaagtctcgagaccggatcctgaatgtcttccacgagctgaacctgaaggatgccatcagctacgtggctgagggagagcgccgcgggtccctggccttcatccgctcccccagcaccgacaacgtggtcaacgtggacttcacgccacgatcgtccaccgtggaggcctctgtctcctacctcctgagagaaaatgtcagcgctgtctgcctggacatgcagtctctggagcagcgacggcggagcatccgggacgcggaggacatggtcacgcaccacacgctacagcagtacctgtacaagccgcggcaggagtacaagcatctgtacagccgacacgagctcacgcccacggaggacgagaaacaggaccgggaaatcttccacaggaccatgcggaagcgcctggagtccttcaagtcgaccaagctggggctcaaccagaacaagaaggcggccaagctgtacaagcgggagcgtgcccagaagcggagaaacagcagcatccccaatgggaagctgcccatggagagccctgcgcagaatttcaccatcaaggagaaagacttggaactttcagacaccgaggagccccccaactatgatgaggagatgagtggggggatcgagttcctggctagtgtcaccaaggacacagcgtccgactcccctgcaggaattgacaaccctgtgttttctccggacgaggccctggaccgcagcctcctggccaggctgccgccctggctgtctcccggggagacggtggtcccctcgcagagggcccgcacgcagattccctactctcccggcaccttctgccgcctgatgcccttccgcctcagcagcaagtccgtggactccttcctgcaggcagacggccccgaggagcggccccccgccgccctccccgagtccacacacatgtgacaccggctccgacacgccgctaaccggccgctcgtccccgcgccacggtccgcccaccgccgccgccgctgtcccagagaaaccccagatccccgcgcggctacagcgaccgcgagccccgagccccgcctcgcccacgccagcgcctgcgcagaactcc

(19) United States
(12) Patent Application Publication
(10) Pub. No.: US 2018/0038873 A1
(43) Pub. Date: Feb. 8, 2018

(54) USE OF NHE3 AS A BIOMARKER FOR RADIATION BIODOSIMETRY

(71) Applicant: UNIVERSITY OF FLORIDA RESEARCH FOUNDATION, INC., Gainesville, FL (US)

(72) Inventors: SADASIVAN VIDYASAGAR, Gainesville, FL (US); PAUL OKUNIEFF, Gainesville, FL (US); ANAND VIDYASAGAR, Gainesville, FL (US)

(21) Appl. No.: 15/549,166

(22) PCT Filed: Feb. 5, 2016
(86) PCT No.: PCT/US2016/016803

§ 371 Date: Aug. 6, 2017

Related U.S. Application Data
(60) Provisional application No. 62/112,467, filed on Feb. 5, 2015.

(51) Int. Cl. G01N 33/68; G01N 33/567; C12Q 1/68

(52) U.S. Cl. CPC G01N 33/6893; C12Q 1/6876; G01N 33/567; C12Q 2600/158; G01N 2800/40

(57) ABSTRACT

Embodiments of the present invention are directed to the use of NHE3 as an early biomarker for radiation biodosimetry. In other embodiments, the present invention relates to the use of NHE3 as a biomarker for determining the absorbed radiation dose in a subject who has been exposed to a known or unknown dose of ionizing radiation. Further embodiments relate to the use of NHE3 as a biomarker for determining effectiveness of a therapy for reducing radiation toxicity. Advantageously, the diagnostic and prognostic assays of the present invention are rapid, sensitive, and non-invasive, rendering it useful in civilian and military industries.

### CROSS-REFERENCE TO A RELATED APPLICATION

This application claims the priority benefit of U.S. Provisional Application Ser. No. 62/112,467, filed Feb. 5, 2015, which is incorporated herein by reference in its entirety.

The Sequence Listing for this application is labeled “SeqList-04FEB16.txt”, which was created on Feb. 4, 2016, and is 11 KB. The entire content is incorporated herein by reference in its entirety.

### BACKGROUND OF INVENTION

Every year approximately one million patients are treated with radiation therapy. Inadvertent exposure is a health risk for workers in certain manufacturing sectors and weapons development. Moderate- and high-level doses can cause a day or two of nausea, vomiting, loss of appetite and diarrhea, which often disappear for about a week before serious health problems emerge. During the latent period, it is difficult to diagnose impending illness. Until now, healthcare providers have been forced to take a wait-and-see approach.

Radiation therapy, a common treatment regime for cancer, can cause severe damage to radiosensitive organs, including the bone marrow, the gastrointestinal (GI) tract, and the lung. Toxic effects of radiation on the gastrointestinal system cause symptoms such as nausea, vomiting, diarrhea, electrolyte imbalance, and dehydration. Radiation can also cause pulmonary injury, leading to pulmonary pneumonitis and fibrosis.

Radiation toxicity not only causes devastating effects on the quality of patient life, but can sometimes even be more life-threatening than the primary tumor or cancer. Therefore, it is important to monitor the severity of radiation toxicity in patients during the course of radiation therapy.

### BRIEF SUMMARY

Sodium hydrogen exchanger isoform 3 (NHE3) is a key transporter responsible for absorbing sodium into the cell in the gastrointestinal tract.

In one embodiment, the present invention pertains to the use of NHE3 as an early biomarker for radiation biodosimetry. In a specific embodiment, the present invention relates to the use of NHE3 as a biomarker for diagnosing the presence of radiation toxicity in a subject who has been exposed to ionizing radiation.

In another embodiment, the present invention relates to the use of NHE3 as a biomarker for determining the absorbed radiation dose in a subject who has been exposed to a known or unknown dose of ionizing radiation.

In a further embodiment, the present invention relates to the use of NHE3 as a biomarker for determining effectiveness of a therapy for reducing radiation toxicity.

Advantageously, the diagnostic and prognostic assays of the present invention are rapid, sensitive, and non-invasive. The present invention is useful in civilian and military applications.

In one embodiment, the present invention provides a method for determining radiation dose absorbed by a subject who has been, or is suspected of having been, exposed to ionizing radiation, wherein the method comprises:

(a) providing a biological sample from a subject who has been, or is suspected of having been, exposed to ionizing radiation;

(b) determining NHE3 expression level in the subject's biological sample; and

(c) determining the radiation dose absorbed by the subject based on the level of expression determined in step (b).

In another embodiment, the present invention provides a method of determining whether a subject has radiation toxicity, wherein the method comprises:

(a) providing a biological sample from a subject who has been, or is suspected of having been, exposed to ionizing radiation;

(b) determining NHE3 expression level in the subject's biological sample; and

(c) comparing the expression level determined in step (b) to a level of NHE3 expression in a normal control;

wherein an increased expression of NHE3 in the subject's biological sample with respect to the control indicates that the subject has radiation toxicity.

In one embodiment, the absorbed radiation dose and/or the presence of radiation toxicity is determined based on the NHE3 expression level in a biological sample. In specific embodiments, the biological sample is a blood sample (such as whole blood, plasma, and serum).

In another embodiment, the method of determining the absorbed radiation dose and/or the present of radiation toxicity further comprises assaying for the expression level(s) of the family of anoctamin proteins (ANO 1-10). See, for example, the invention disclosed in PCT Publication No. WO 2014/105249, which is incorporated herein, in its entirety, by reference.

In a further embodiment, expression level of NHE3 can be used as a secondary endpoint to determine mechanisms of action and/or pharmacodynamic (PD) effects of an agent for reducing radiation toxicity.

### BRIEF DESCRIPTION OF SEQUENCES

SEQ ID NO:1 is the amino acid sequence of a human sodium hydrogen exchanger isoform 3 protein (GenBank Accession No. NP_004165).

SEQ ID NO:2 is the nucleic acid sequence of a human sodium hydrogen exchanger isoform 3 mRNA transcript (GenBank Accession No. NM_004174).

### DETAILED DISCLOSURE

In one embodiment, the present invention pertains to the use of NHE3 as an early biomarker for radiation biodosimetry. In a specific embodiment, the present invention relates to the use of NHE3 as a biomarker for diagnosing the presence of radiation toxicity in a subject who has been exposed to ionizing radiation.

In another embodiment, the present invention relates to the use of NHE3 as a biomarker for determining the absorbed radiation dose in a subject who has been exposed to a known or unknown dose of ionizing radiation.

In further embodiment, the present invention relates to the use of NHE3 as a biomarker for determining effectiveness of a therapy for reducing radiation toxicity. In one embodiment, the expression level of NHE3 can be used as a secondary endpoint to determine mechanisms of action and/or pharmacodynamics (PD) effects of an agent for reducing radiation toxicity.

After irradiation, glucose transport is partially or completely down-regulated in a dose-dependent manner. As a result, oral glucose intake after irradiation would activate calcium-activated electrogenic chloride secretion, thereby resulting in secretory diarrhea. Western blot analysis of the small intestinal mucosa of mice exposed to irradiation shows increased NHE3 expression even on day 6 post-irradiation. NHE3 expression level is also increased in the membrane of red blood cells (RBCs) after irradiation.

In accordance with the subject invention, it has been found that irradiation decreases NHE3 expression along the brush border membrane of the villous epithelial cells, resulting in reduced sodium and chloride absorption and therefore fluid absorption. Reduced electrolyte and fluid absorption leads to increased fluid in the gut lumen, stool volume and therefore diarrhea. Since the decrease in NHE protein levels with irradiation in RBC and/or WBC membranes also parallels its level on villous epithelial cells, blood cell membranes can be used as a surrogate marker for acute gastrointestinal toxicity.

The diagnostic test of the subject invention can be used to help predict the onset and severity of radiation-induced gastrointestinal toxicity in a person who has been exposed either as part of cancer treatment or as part of accidental or intentional radiation exposure. Using a simple test that measures NHE3 levels in, for example, the blood, it is possible to assess an individual's radiation dose or if a patient is developing radiation toxicity. This information allows doctors to adjust treatment regimes, lessening side effects and preventing deaths. Research labs, hospitals, biodefense facilities and other organizations that deal with ionizing radiation will benefit greatly from this easy-to-use diagnostic blood test.

Advantageously, the diagnostic and prognostic assays of the present invention are rapid, sensitive, and non-invasive. The present invention can be useful in civilian and military industries.

The term “subject,” as used herein, describes an organism, including mammals such as primates. Mammalian species that can benefit from the disclosed methods of treatment include, but are not limited to, apes, chimpanzees, orangutans, humans, monkeys; and other animals such as dogs, cats, horses, cattle, pigs, sheep, goats, chickens, mice, rats, guinea pigs, and hamsters. In one embodiment, the subject is a human.

The term “biological sample,” as used herein, includes, but is not limited to, a sample containing tissues, cells, and/or biological fluids isolated from a subject. Examples of biological samples include, but are not limited to, tissues, cells, biopsies, blood, lymph, serum, plasma, urine, saliva, and tears. In one embodiment, the biological sample contains red blood cells.

In one embodiment, the present invention provides a method for determining radiation dose absorbed by a subject who has been, or is suspected of having been, exposed to ionizing radiation, wherein the method comprises:

(a) providing a biological sample from a subject who has been, or is suspected of having been, exposed to radiation (such as ionizing radiation);

(b) determining expression level of an NHE3 in the subject's biological sample; and

(c) determining the radiation dose absorbed by the subject based on the level of expression determined in step (b).

In another embodiment, the present invention provides a method of determining whether or not a subject has radiation toxicity, wherein the method comprises:

(a) providing a biological sample from a subject who has been, or is suspected of having been, exposed to radiation (such as ionizing radiation);

(b) determining expression level of an NHE3 in the subject's biological sample; and

(c) comparing the expression level determined in step (b) to a level of an NHE3 expression in a normal control;

wherein an increased expression of an NHE3 in the subject's biological sample with respect to the control indicates that the subject has radiation toxicity.

In one embodiment, an increased expression of an NHE3 in the subject's biological sample with respect to the control indicates that the subject has radiation-induced acute gastrointestinal toxicity.

In a further embodiment, the present invention provides a method of determining whether a subject has developed radiation toxicity during the course of radiation therapy, wherein the method comprises:

(a) providing a biological sample from a subject who has been prescribed radiation therapy at a predetermined dose;

(b) before radiation therapy, determining expression level of an NHE3 in a biological sample of the subject;

(c) providing radiation therapy to the subject at the predetermined dose;

(d) determining expression level of an NHE3 in the subject's biological sample after the subject has been exposed to radiation at the predetermined dose;

(e) comparing the expression level determined in step (d) to the NHE3 expression level determined in step (b); and

(f) if the level of NHE3 expression determined in (d) is at least 105%, 110%, 115%, 120%, 125%, 130%, 135%, 140%, 150%, 160%, 170%, 180%, 190%, 200%, 250%, 300%, 400%, or 500% of the NHE3 expression level determined in step (b), then the subject has radiation toxicity.

In certain embodiments, the present invention can be used to determine the absorbed radiation dose and/or determine the presence of radiation toxicity 1, 2, 3, 4, 5, 6, 7, 8, 9, 10, 11, 12, 13, or 14 days after the subject has received, or is suspected of receiving, irradiation.

In certain embodiments, NHE3 expression level is determined using a biological sample obtained no more than 1, 2, 3, 4, 5, 6, 7, 8, 9, 10, 11, 12, 13, or 14 days after the subject has received, or is suspected of receiving, ionizing radiation.

In certain embodiments, NHE3 expression level is determined using a biological sample obtained no more than 1, 2, 3, 4, 5, 6, 7, 8, 9, 10, 11, 12, 13, or 14 days after the subject has received, or is suspected of receiving, radiation at a dose of at least 0.5 Gy or higher (including, but not limited to, at least 2, 3, 4, 5, 6, 7, 8, 9, 10, 15, 20, 25, 30, 35, 40, 45, 50, 55, 60, 65, 70, 80, 85, and 90 Gy).

In one embodiment, the absorbed radiation dose and/or the presence of radiation toxicity is determined based on the expression level in a biological sample. In one embodiment, the biological sample is a blood sample (such as whole blood, plasma, and serum). In one embodiment, the level of an NHE3 in the membranes of RBCs of a subject is determined.

In a further embodiment, the NHE3 expression level in a subject is determined at multiple time points to determine whether the subject has radiation toxicity, to monitor the severity of radiation toxicity, and/or to determine the treatment effects of a therapeutic regime for reducing radiation toxicity.

In one embodiment, the subject has been exposed to radiation during the course of radiation therapy for tumor or cancer. In another embodiment, the subject has been, or is suspected of having been, exposed to ionizing radiation by accident.

In certain embodiments, the subject has been exposed to radiation (such as via prescription during ionizing radiation therapy) or is suspected of having been exposed to radiation (such as by accidental exposure to ionizing radiation) at a dose of at least 0.5, 1, 2, 3, 4, 5, 6, 7, 8, 9, 10, 15, 20, 25, 30, 35, 40, 45, 50, 55, 60, 65, 70, 80, 85, or 90 Gy. In certain embodiments, the subject has been exposed to radiation (such as via prescription during ionizing radiation therapy) or is suspected of having been exposed to radiation (such as by accidental exposure to ionizing radiation) at a dose of at least 0.1, 0.3, 0.5, 0.7, 0.9, 1.0, 1.1, 1.2, 1.3, 1.4, 1.5, 1,6, 1.7, 1.8, 1.9, 2.0, 2.1, 2.2, 2.3, 2.4, 2.5, 2,7, 3.0, 3.2, 3.5, or 4.0 Gy in one day.

In a further embodiment, the present invention provides a method of providing a toxicity-monitored radiation therapeutic regime, wherein the method comprises:

(a) providing a biological sample from a subject who has been exposed to radiation therapy at a predetermined dose;

(b) determining NHE3 expression level in the subject's biological sample after the subject has been exposed to radiation at the predetermined dose;

(c) comparing the expression level determined in step (b) to a level of NHE3 expression in a normal control;

(d) if the level of NHE3 expression determined in (b) is greater than control, then prescribing additional radiation at a dose lower than the predetermined dose, discontinuing radiation therapy for at least 1 day or any days longer than 1 day (including, but not limited to, at least 2 days, 3 days, 4 days, 5 days, 10 days, 15 days, 30 days, 60 days, 90 days, and 180 days), or prescribing a therapy that reduces radiation-induced toxicity (such as radiation-induced acute gastrointestinal toxicity); and

if the level of NHE3 expression determined in (b) is no greater than the control, then continuing radiation therapy at a dose identical to, or higher than, the predetermined dose.

In a further embodiment, the present invention provides a method of providing a toxicity-monitored radiation therapeutic regime, wherein the method comprises:

(a) providing a biological sample from a subject who has been prescribed radiation therapy at a predetermined dose;

(b) before radiation therapy, determining NHE3 expression level in a biological sample of the subject;

(c) providing radiation therapy to the subject at the predetermined dose;

(d) determining NHE3 expression level in the subject's biological sample after the subject has been exposed to radiation at the predetermined dose;

(e) comparing the expression level determined in step (d) to the NHE3 expression level determined in step (b);

(f) if the level of NHE3 expression determined in (d) is at least 105%, 110%, 115%, 120%, 125%, 130%, 135%, 140%, 150%, 160%, 170%, 180%, 190%, 200%, 250%, 300%, 400%, or 500% of the NHE3 expression level determined in step (b), then prescribing a second radiation dose lower than the predetermined dose, discontinuing radiation therapy for at least 1 day or any days longer than 1 day (including, but not limited to, at least 2 days, 3 days, 4 days, 5 days, 10 days, 15 days, 30 days, 60 days, 90 days, and 180 days), or prescribing a therapy that reduces radiation-induced toxicity (such as radiation-induced acute gastrointestinal toxicity); and

if the level of NHE3 expression determined in (d) is no greater than 105%, 110%, 115%, 120%, 125%, 130%, 135%, 140%, 150%, 160%, 170%, 180%, 190%, 200%, 250%, 300%, 400%, or 500% of the NHE3 expression level determined in step (b), then continuing the prescribed radiation dose.

In certain embodiments, in the course of providing a toxicity-monitored radiation therapeutic regime, NHE3 expression level is determined 1, 2, 3, 4, 5, 6, 7, 8, 9, 10, 11, 12, 13, or 14 days after the subject has received the predetermined radiation dose. The NHE3 expression level can be determined at multiple time points. In certain embodiments, therapies that reduce radiation-induced toxicity (such as radiation-induced acute gastrointestinal toxicity) include, but are not limited to, oral rehydration compositions, and compositions disclosed in PCT/US2011/053265, which is hereby incorporated by reference in its entirety.

In one embodiment, the absorbed radiation dose and/or the presence of radiation toxicity is determined based on expression level of NHE3.

In another embodiment, the method of determining the absorbed radiation dose and/or the presence of radiation toxicity further comprises assaying for the expression level(s) of the family of anoctamin proteins (ANO 1-10). See, for example, the invention disclosed in PCT Publication WO 2014/105249.

The level of NHE3 expression can be determined based on mRNA levels or protein levels. Determination of NHE3 expression can be made qualitatively, semi-quantitatively, or quantitatively. Sequences of NHE3 proteins and mRNAs of a variety of mammalian species are publicly available and can be obtained from, for example, the GenBank database. One of ordinary skill in the art, having the benefit of the present disclosures, can easily use NHE3 protein and nucleic acid sequences of a mammalian species of interest to practice the present invention.

In one embodiment, the control level of NHE3 expression is determined by measuring NHE3 expression in a healthy population that has not been exposed to radiation (such as ionizing radiation) and/or does not have acute or long term side effects caused by irradiation.

Methods for determining NHE3 expression level are well known in the art, including but not limited to, Western blot, enzyme-linked immunosorbent assay (ELISA), immunoprecipitation, polymerase chain reaction (PCR) methods including reverse transcription polymerase chain reaction (RT-PCR), nucleic acid hybridization, and any combination thereof. In a preferred embodiment, the NHE3 expression level is determined using ELISA.

A contacting step in the assay (method) of the invention can involve contacting, combining, or mixing the biological sample and a solid support, such as a reaction vessel, microbeads, microvessel, tube, microtube, well, multi-well plate, or other solid support.

An antibody that specifically recognizes, or specifically binds to, NHE3 proteins can be in any of a variety of forms, including intact immunoglobulin molecules, fragments of immunoglobulin molecules such as Fv, Fab and similar fragments; multimers of immunoglobulin molecules (e.g., diabodies, triabodies, and bi-specific and tri-specific antibodies, as are known in the art; see, e.g., Hudson and Kortt, J. Immunol. Methods, 231:177, 1999); fusion constructs containing an antibody or antibody fragment; and human or humanized immunoglobulin molecules or fragments thereof.

“Specific binding” or “specificity” refers to the ability of a protein to detectably bind an epitope presented on a protein or polypeptide molecule of interest, while having relatively little detectable reactivity with other proteins or structures. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific target molecule versus nonspecific binding to other irrelevant molecules.

Antibodies within the scope of the invention can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

Antibodies of the present invention include polyclonal and monoclonal antibodies. The term “monoclonal antibody,” as used herein, refers to an antibody or antibody fragment obtained from a substantially homogeneous population of antibodies or antibody fragments (i.e., the individual antibodies within the population are identical except for possible naturally occurring mutations that may be present in a small subset of the antibody molecules).

In one embodiment, the level of NHE3 protein expression is determined by contacting the biological sample with an antibody that specifically recognizes, or specifically binds to, an NHE3 protein; and detecting the complex formed between the antibody and the NHE3 protein.

The level of NHE3 expression can be determined based on NHE3 mRNA level. In one embodiment, the NHE3 mRNA level can be determined by a method comprising contacting the biological sample with a polynucleotide probe that comprises a nucleic acid sequence that specifically binds to, or hybridizes under stringent conditions with, an NHE3 mRNA; and detecting the complex formed between the polynucleotide probe and the NHE3 mRNA.

As used herein, “stringent” conditions for hybridization refers to conditions wherein hybridization is typically carried out overnight at 20-25° C. below the melting temperature (Tm) of the DNA hybrid in 6×SSPE, 5× Denhardt's solution, 0.1% SDS, 0.1 mg/ml denatured DNA. The melting temperature, Tm, is described by the following formula (Beltz et al., 1983):

Tm=81.5 C+16.6 Log[Na+]+0.41(% G+C)−0.61(% formamide)−600/length of duplex in base pairs.

Washes are carried out as follows:

(1) Twice at room temperature for 15 minutes in 1×SSPE, 0.1% SDS (low stringency wash).

(2) Once at Tm—20 C for 15 minutes in 0.2×SSPE, 0.1% SDS (moderate stringency wash).

In one embodiment, the NHE3 mRNA level can be determined by polymerase chain reaction methods. Polymerase chain reaction (PCR) is a process for amplifying one or more target nucleic acid sequences present in a nucleic acid sample using primers and agents for polymerization and then detecting the amplified sequence. The extension product of one primer when hybridized to the other becomes a template for the production of the desired specific nucleic acid sequence, and vice versa, and the process is repeated as often as is necessary to produce the desired amount of the sequence. The skilled artisan, to detect the presence of a desired sequence (U.S. Pat. No. 4,683,195), routinely uses polymerase chain reaction.

A specific example of PCR that is routinely performed by the skilled artisan to detect desired sequences is reverse transcript PCR (RT-PCR; Saiki et al., Science, 230:1350, 1985; Scharf et al., Science, 233:1076, 1986). RT-PCR involves isolating total RNA from biological fluid, denaturing the RNA in the presence of primers that recognize the desired nucleic acid sequence, using the primers to generate a cDNA copy of the RNA by reverse transcription, amplifying the cDNA by PCR using specific primers, and detecting the amplified cDNA by electrophoresis or other methods known to the skilled artisan.

Samples and/or NHE3-specific binding agents may be arrayed on a solid support, or multiple supports can be utilized, for multiplex detection or analysis. “Arraying” refers to the act of organizing or arranging members of a library (e.g., an array of different samples or an array of devices that target the same target molecules or different target molecules), or other collection, into a logical or physical array. Thus, an “array” refers to a physical or logical arrangement of, e.g., biological samples. A physical array can be any “spatial format” or “physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding agents can be plated or otherwise deposited in microtitered, e.g., 96-well, 384-well, or 1536-well plates (or trays). Optionally, NHE3-specific binding agents may be immobilized on the solid support.

In another embodiment, the present invention provides a method for screening for a therapeutic agent that reduces radiation toxicity, wherein the method comprises:

(a) providing a population of cells that have been exposed to radiation (such as ionizing radiation) and have an increased expression of NHE3, and optionally, determining a first level of NHE3 expression in the population of cells exposed to radiation (such as ionizing radiation);

(b) contacting the population of cells with a candidate therapeutic agent for reducing radiation toxicity;

(c) after step (b), determining NHE3 expression level in the population of cells contacted with the candidate therapeutic agent; and

(d) selecting the candidate agent that reduces the level of NHE3 expression as the therapeutic agent that reduces radiation toxicity.

In another embodiment, the present invention provides a method for identifying an agent that increases radiation toxicity, wherein the method comprises:

(a) providing a population of cells that have been exposed to radiation (such as ionizing radiation) and have an increased expression of NHE3, and optionally, determining a first level of NHE3 expression in the population of cells exposed to radiation (such as ionizing radiation);

(b) contacting the population of cells with a candidate agent;

(c) after step (b), determining NHE3 expression level in the population of cells contacted with the candidate agent; and

(d) identifying the candidate agent that increases the level of NHE3 expression, when compared to the first level of NHE3 expression, as an agent that increases radiation toxicity.

In a further embodiment of the screening method, the candidate agent is contacted with a population of cells of a subject who has been exposed to radiation (such as ionizing radiation).

### Kits

The present invention provides kits comprising the required elements for detecting NHE3.

In one embodiment, the present invention provides a kit for determining whether the subject has radiation toxicity, for determining the absorbed radiation dose, for monitoring the severity of radiation toxicity, and/or for determining the treatment effects of a therapeutic regime for reducing radiation toxicity.

In certain specific embodiments, the kit comprises an application zone for receiving a biological sample (such as a blood sample); a labeling zone containing a binding agent that binds to an NHE3 protein or mRNA in the sample; and a detection zone where NHE3-bound binding agent is retained to give a signal, wherein the signal given for a sample of a subject with an NHE3 level greater than a control level is different from the signal given for a sample of a subject with an NHE3 level lower than a control level.

In one embodiment, the kit comprises an NHE3-binding agent including, an antibody that specifically recognizes, or specifically binds to, an NHE3 protein; a polynucleotide probe that comprises a nucleic acid sequence that specifically binds to, or hybridizes under highly stringent condition to, an NHE3 mRNA; and a primer set that amplifies an NHE3 mRNA.

Preferably, the kits comprise a container for collecting samples, such as blood samples, from a subject, and an agent for detecting the presence or the level of NHE3 in the sample. The agent may be any binding agent specific for NHE3, including, but not limited to, antibodies, aptamers, nucleic acid probes, and primers. The components of the kit can be packaged either in aqueous medium or in lyophilized form.

As indicated above, kits of the invention include reagents for use in the methods described herein, in one or more containers. The kits may include specific internal controls, and/or probes, buffers, and/or excipients, separately or in combination. Each reagent can be supplied in a solid form or liquid buffer that is suitable for inventory storage. Kits may also include means for obtaining a sample from a host organism or an environmental sample.

Kits of the invention can be provided in suitable packaging. As used herein, “packaging” refers to a solid matrix or material customarily used in a system and capable of holding within fixed limits one or more of the reagent components for use in a method of the present invention. Such materials include glass and plastic (e.g., polyethylene, polypropylene, and polycarbonate) bottles, vials, paper, plastic, and plastic-foil laminated envelopes and the like. Preferably, the solid matrix is a structure having a surface that can be derivatized to anchor an oligonucleotide probe, primer, molecular beacon, specific internal control, etc. Preferably, the solid matrix is a planar material such as the side of a microtiter well or the side of a dipstick. In certain embodiments, the kit includes a microtiter tray with two or more wells and with reagents including primers, probes, specific internal controls, and/or molecular beacons in the wells.

Kits of the invention may optionally include a set of instructions in printed or electronic (e.g., magnetic or optical disk) form, relating information regarding the components of the kits and/or how to make various determinations (e.g., NHE3 levels, comparison to control standards, etc.). The kit may also be commercialized as part of a larger package that includes instrumentation for measuring other biochemical components.

All patents, patent applications, provisional applications, and publications referred to or cited herein are incorporated by reference in their entirety, including all figures and tables, to the extent they are not inconsistent with the explicit teachings of this specification.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The term “consisting essentially of,” as used herein, limits the scope of the ingredients and steps to the specified materials or steps and those that do not materially affect the basic and novel characteristic(s) of the present invention, i.e., compositions and methods for decellularization of tissue grafts. For instance, by using “consisting essentially of,” the compositions do not contain any unspecified ingredients including, but not limited to, surfactants that have a direct beneficial or adverse effect on decellularization of tissue.

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method for determining radiation dose absorbed by a subject who has been, or is suspected of having been, exposed to ionizing radiation, wherein the method comprises:
(a) providing a biological sample from a subject who has been, or is suspected of having been, exposed to ionizing radiation;
(b) determining expression level of NHE3 in the subject's biological sample; and
(c) determining the radiation dose absorbed by the subject based on the level of expression determined in step (b).

2. The method according to claim 1, wherein the biological sample is a blood sample.

3. The method according to claim 1, wherein the NHE3 expression level in the subject's biological sample is determined by Western blot, enzyme-linked immunosorbent assay (ELISA), immunoprecipitation, reverse transcription polymerase chain reaction (RT-PCR), nucleic acid hybridization, or a combination thereof.

4. The method according to claim 3, wherein the NHE3 expression level in the subject's biological sample is determined by ELISA.

5. The method according to claim 1, wherein the subject has been, or is suspected of having been, exposed to ionizing radiation at a dose of at least 1 Gy.

6. The method according to claim 1, wherein the subject is a human.

7. The method, according to claim 1, which further comprises assaying for the expression level of an anoctamin protein.

8. A method of determining whether a subject has radiation toxicity, wherein the method comprises:
(a) providing a biological sample from a subject who has been, or is suspected of having been, exposed to ionizing radiation;
(b) determining NHE3 expression level in the subject's biological sample; and
(c) comparing the expression level determined in step (b) to a level of NHE3 expression in a normal control;
wherein an increased expression of NHE3 in the subject's biological sample with respect to the control indicates the subject has radiation toxicity.

9. The method according to claim 8, wherein the biological sample is a blood sample.

10. The method according to claim 8, wherein the NHE3 expression level in the subject's biological sample is determined by Western blot, enzyme-linked immunosorbent assay (ELISA), immunoprecipitation, reverse transcription polymerase chain reaction (RT-PCR), nucleic acid hybridization, or a combination thereof.

11. The method according to claim 10, wherein the NHE3 expression level in the subject's biological sample is determined by ELISA.

12. The method according to claim 8, wherein the subject has been, or is suspected of having been, exposed to ionizing radiation at a dose of at least 1 Gy.

13. The method according to claim 8, wherein the subject is a human.

14. The method, according to claim 8, which further comprises assaying for the expression level of an anoctamin protein.

15. The method according to claim 8, wherein the subject had received a predetermined dose of ionizing radiation in a radiation therapeutic regime, and wherein the method further comprises providing a toxicity-monitored radiation therapeutic regime in the subject comprising the steps of:
(d) if the level of NHE3 expression determined in (b) is greater than control, then prescribing additional radiation at a dose lower than the predetermined dose or discontinuing the radiation therapy; and
if the level of NHE3 expression determined in (b) is no greater than the control, then continuing the radiation therapy at the predetermined dose.

16. The method according to claim 8, wherein the subject had received a predetermined dose of ionizing radiation in a radiation therapeutic regime, and wherein the method further comprises providing a toxicity-monitored radiation therapeutic regime in the subject comprising the steps of:
before the subject receives the predetermined dose of ionizing radiation in the radiation therapeutic regime, determining NHE3 expression level in a biological sample of the subject;
if the level of NHE3 expression determined in (b) is greater than 105% of the NHE3 expression level of the subject before the subject receives the predetermined dose of ionizing radiation, then prescribing additional radiation at a dose lower than the predetermined dose or discontinuing radiation therapy; and
if the level of NHE3 expression determined in (b) is no greater than 105% of the NHE3 expression level of the subject before the subject receives the predetermined dose of ionizing radiation, then continuing the radiation therapy at the predetermined dose.

17. A method for screening for a therapeutic agent that reduces radiation toxicity, wherein the method comprises:
(a) providing a population of cells exposed to ionizing radiation and having an increased expression of NHE3, and determining a first level of NHE3 expression in the population of cells exposed to ionizing radiation;
(b) contacting the population of cells with a candidate therapeutic agent for reducing radiation toxicity;
(c) after step (b), determining NHE3 expression level in the population of cells contacted with the candidate therapeutic agent; and
(d) selecting the candidate agent that reduces the level of NHE3 expression as the therapeutic agent that reduces radiation toxicity.

18. The method according to claim 17, wherein the candidate agent is contacted with a population of cells of a subject who has been exposed to ionizing radiation.

19. The method, according to claim 17, which further comprises assaying for the expression level of an anoctamin protein.
